(19) United States
(12) Patent Application Publication
(10) Pub. No.: US 2016/0106508 A1
(43) Pub. Date: Apr. 21, 2016

(54) STRUCTURE FOR SUPPORTING A MEDICAL DEVICE ON A PATIENT DURING SURGICAL PROCEDURES

(71) Applicant: Vanderbilt University, Nashville, TN (US)

(72) Inventors: Ray Lathrop, Nashville, TN (US); Robert J. Webster, Nashville, TN (US); Hunter Gilbert, Nashville, TN (US); Raul Wirz Gonzalez, Nashville, TN (US)

(21) Appl. No.: 14/884,143

(22) Filed: Oct. 15, 2015

Related U.S. Application Data
(60) Provisional application No. 62/064,126, filed on Oct. 15, 2014.
(60) Provisional application No. 62/076,790, filed on Nov. 7, 2014.

(51) Int. Cl. A61B 19/00

(52) U.S. Cl. CPC A61B 19/203; A61B 19/201; A61B 2019/207; A61B 2019/262

(57) ABSTRACT

An apparatus for supporting a medical device on a patient includes a support structure adapted to be fitted to the patient's body. The medical device is connectable to the support structure. At least one mounting pad is connected to the support structure and is adapted to engage the patient's anatomy. The at least one mounting pad includes a frictional jamming structure configured so that, in a non-actuated condition of the mounting pad, the mounting pad conforms to the patient's anatomy and, in an actuated condition of the mounting pad, the mounting pad becomes rigid in its conformed shape.

### RELATED APPLICATIONS

This application claims priority from U.S. Provisional Patent Application Nos. 62/064,126, filed Oct. 15, 2014 and 62/076,790, filed Nov. 7, 2014, the subject matter of which is incorporated herein by reference in its entirety.

### STATEMENT REGARDING FEDERALLY SPONSORED RESEARCH

This invention was made with government support under grant No. R01-4224511091 awarded by the National Institute of Health. The Government has certain rights in this invention.

### TECHNICAL FIELD

The present invention relates to surgical procedures that employ the use of patient mounted structures for the purpose of providing surgical tool support and/or assisting in image-based guidance. More particularly, the present invention relates to a structure for supporting a medical device on a patient during a surgical procedure, wherein the support structure that incorporates granular jamming mounting pads to secure the support structure to the patient.

### BACKGROUND

Patient Mounted Surgical Tools

Some surgical procedures employ the use of surgical tools that are mounted on the patient's anatomy. For example, some surgical procedures, such as endonasal procedures, can employ the use of robotic or manually operated tools, such as endoscopes for viewing the surgical field or probes having various tool ends (e.g., forceps, graspers, cutters, needles, drug delivery devices, ablation elements, drills, etc.) that are mounted on the patient's head. Patient mounted surgical tools require a stable, reliable, and rigid mounting structure to ensure that the tool mount does not move relative to the patient in during use. These relative movements are undesirable because they can lead to errors, especially in the case of surgical robots, whose accuracy and precision depend on maintaining stable positioning of the mounting structure.

Image Guided Surgery

During image guided surgery of the skull, the location of the head is tracked by an optical tracking system that employs means, such as a stereoscopic camera, to determine the location of optical tracking fiducials. Tracking fiducials mounted to the patient's head allow the tracking system to identify and follow the location of the patient's head. Fiducials mounted to a surgical tool, such as a probe, allow the tracking system to identify and follow the location of the tool, particularly the tip of the tool. It is very important that, once registered, the positions of the fiducials relative to the structures to which they are mounted (i.e., the head or the surgical tool) do not change. If this happens, the resulting registration errors will cause the optical tracking system to provide erroneous indications of the location of the tool relative to the patient anatomy.

Fiducials are connected to the surgical tool via a rigid mechanical connection that is extremely reliable and therefore not prone to post-registration movement that produces registration errors. Current methods and devices for attaching fiducials to the patient's head/skull are less reliable and can be prone to post-registration movement relative to the patient's head. This relative movement produces registration errors that affect the accuracy of the image navigation system and can compromise the safety of the patient. It is therefore important to minimize or eliminate relative motion between the head/skull and the tracking fiducials attached thereto. While surgically mounting the fiducial directly to the patient's bone structure is effective in helping to minimize registration errors, this attachment method is not ideal since it necessitates an additional surgical procedure, which introduces unwanted complexity and risks.

Accurate registration of the images to the patient anatomy is essential to enable the optical tracking system to accurately represent tool position with respect to the patient anatomy in general and, more specifically, to target(s) in the anatomy. Target. Registration Error (“TRE”) is related to Fiducial Localization Error (“FLE”) and Fiducial Registration Error (“FRE”). This relationship, however, is based on the assumption that there is a rigid connection between the bone of the patient's head and the fiducials tracked by the image guidance system. Theoretical calculations show that, if used properly, clinical image guidance systems should be expected to provide very low TRE, i.e., should be highly accurate. Yet, anecdotally, surgeons report that sometimes errors are much higher than expected. Errors in these cases can reach several millimeters at the skull base. These errors are not insignificant given that surgeons often must work within closer proximity to delicate anatomical structures, such as the optic nerves and carotid arteries.

There are several different registration techniques that can be utilized in conventional clinical image guidance systems. Examples of these techniques include stereotactic frame registration, point-based registration, and surface-based registration. Stereotactic frames employ rigid frames that are fixed to the patient typically via clamps and bars. Stereotactic frames are known to have a TRE of 1-2 mm. Bone-screw-based fiducial markers are surgically fixed directly to the patient's bone structure and are known to have a TRE of 1-1.5 mm. Skin-affixed fiducials are connected to the patient's skin. e.g., via adhesive, and are known to have a TRE of 1.3-4 mm. Surface-based registration employs surface scanning techniques to establish registration and is known to have a TRE of 2-5 mm.

In endonasal surgery, stereotactic frames and bone screw-based fiducial markers are typically avoided due to their invasiveness. Due to this, non-invasive surface-based registration is employed. For example, the Kolibri® system manufactured by Brainlab AG of Feldkirchen, Germany, is an image-guided system that uses points collected from the brow and bridge of the nose (i.e. a “browscan”) for registration. The location of the patient's head as well as the location(s) of the surgical tools are then tracked in real time by a stereo camera system that observes fiducials attached to each throughout the surgery. This enables the image guidance system to perform its function of showing the location of the tip of each tracked surgical tool with respect to registered preoperative images during the surgery.

According to this surface-based registration procedure, a fiducial marker is supported on a rigid body that is attached to the skin of the patient's forehead using double sided foam tape and an elastic strap that wraps around the patient's head. A laser scanner is used to scan the surface of the brow which is then used by the optical tracking system to establish the shape and location of the brow and thus the skull. At the same time the scan is being performed, the optical tracking system locates the fiducial. It is assumed the rigid body and brow will not move relative to one another. Thus, the location of the skull is based on the location of the fiducial once the scan is complete, i.e., the fiducial is registered relative to the skull.

Any post-registration movement of the fiducial relative to the patient's head results in a TRE. This can occur, for example, where hospital staff accidentally bump into the rigid body, or where the staff lacks the training and understanding to appreciate the delicacy of the system when handling the patient and/or the equipment. Since the tracking rigid body is typically affixed to the head using the aforementioned adhesive and elastic headband, care must be taken to avoid displacing the skin or bumping the rigid body. Prior work has shown that human skin can shift in the range of 1.3-13.1 mm, with a mean of 5.34±2.65 mm under load. A previous report of 35 sinus surgery cases found movement of the headband in 14% of cases, indicating that improved fixation methods are needed. Registration accuracy depends strongly on careful fixation of the rigid body, and thus the tracking fiducial, to the patient.

### SUMMARY

According to one aspect of the invention, a non-invasive means for fixing a support structure for a surgical tool or tracking fiducial to a patient includes a mounting elements whose configuration is based on the physical properties of jammed systems. More specifically, this approach employs the use of a jammed granular substance in mounting pads that are used to help secure the support structure to the patient. In one example, the granular jamming mounting pads are used to help secure the support structure to a patient's head.

Under this approach, granular jamming pads include a granular substance, such as granular silica, in a container or confinement constructed of a flexible deformable material, such as a plastic/polymer material or a rubber material, that is both strong and substantially airtight. For instance, the granular substance can have a consistency similar to that of coffee grounds. The jamming pads are arranged on a support structure, such as a frame or helmet, that in turn supports a surgical tool (in the case of a surgical tool mount) or a rigid body and target fiducials (in the case of a target fiducial mount). When the support structure is positioned on the patient with the granular jamming pads engaging the patient's head, the granular substance acts like a fluid and the pads conform to the specific anatomy of the head. Held in position using straps or other suitable means, a vacuum is drawn on the pads, which collapses the pad container and jams the granular substance together, causing it to harden and act like a solid.

The jamming pads, when hardened, are custom fitted to the exact contour of the patient's head. Thus, by arranging the pads to engage the patient's head in a manner such that the pads encompass or encircle a substantial portion of its surface, the hardened pads can create an interference fit with the head that, together with the securing forces applied by the straps, maintains the support structure, and any surgical tools or target fiducials mounted thereon, in a precisely fitted and maintained position on the patient's head.

According to one aspect, an apparatus for supporting a medical device on a patient includes a support structure adapted to be fitted to the patient's body. The medical device is connectable to the support structure. At least one mounting pad is connected to the support structure and is adapted to engage the patient's anatomy. The at least one mounting pad includes a frictional jamming structure configured so that, in a non-actuated condition of the mounting pad, the mounting pad conforms to the patient's anatomy and, in an actuated condition of the mounting pad, the mounting pad becomes rigid in its conformed shape.

According to another aspect, the apparatus can include a flexible elongated member, such as a strap, for helping to apply a force to the support structure that compresses the at least one mounting pad between the support structure and the patient.

According to another aspect, the at least one mounting pad can include a casing that at least partially surrounds the jamming structure. The apparatus can further include a vacuum source operatively connected to the at least one mounting pad and being operative to draw a vacuum in the casing of the at least one mounting pad.

According to another aspect, the jamming structure can be a volume of granular material.

According to another aspect, the granular material can include at least one of granular silica, polymer beads of varying shapes and configurations, and coffee grounds.

According to another aspect, the casing can be constructed out of at least one of a plastic material, a polymer material, and a natural or synthetic rubber material. The casing can have a latex rubber construction.

According to another aspect, the support structure can be adapted for connection to the patient's head.

According to another aspect, the support structure can include a shell in the general form of a helmet.

According to another aspect, the support structure can include a frame comprising a cross member having an adjustable width, and side plates connected to opposite ends of the cross member via a hinged connection.

According to another aspect, the support structure can include a semi-rigid plate adapted to be at least partially wrapped around the patient's head, and a strap connected to opposite ends of the plate for securing the plate to the patient's head.

According to another aspect, the support structure can include a strap to which the mounting pads are connected, the strap being adapted to wrap around the circumference of the patient's head.

According to another aspect, the medical device can include a rigid body for supporting one or more target fiducials for registering the patient's head in an image guided surgical procedure.

According to another aspect, the medical device can include a surgical tool for performing a surgical procedure on the patient.

According to another aspect, the apparatus can include a bracket connected to the support structure, the bracket being configured to cooperate with a patient support structure to help stabilize the position of the patient.

According to another aspect, the support structure can include C-shaped frame for extending laterally around the patient's head across the forehead and wrapping at least partially around the back of the head. The support device can also include a top member connected to the middle of the C-shaped frame, the top member extending arcuately up and over the top of the patients head, ends of the C-shaped frame and a free end of the top member being interconnected by a flexible elongated member.

According to another aspect, the apparatus can include at least one device mount connected to the support structure, the at least one device mount being adapted to provide a releasable connection with the medical device.

According to another aspect, the jamming structure can include a substrate and a plurality of sheets supported by the substrate and arranged in an adjacent and overlying manner. The sheets can extend generally vertically from the substrate. The sheets can extend generally horizontally from one or more posts mounted to the substrate. The sheets can be sheets of paper or plastic.

### DRAWINGS

FIG. 1 is a front view of a support apparatus according to an example embodiment of the invention.

FIG. 2 is a sectional view of the apparatus taken generally along line 2-2 in FIG. 1.

FIGS. 3A-3C are schematic illustrations of a portion of the apparatus in different conditions.

FIG. 4 is a front view of the apparatus of FIGS. 1-2 illustrating an additional implementation.

FIG. 5 is a front view of a support apparatus according to an additional example embodiment of the invention.

FIG. 6 is a side view of the apparatus illustrated in FIG. 5.

FIG. 7 is a front view of the apparatus of FIGS. 5-6 illustrating an additional implementation.

FIG. 8 is a perspective view of a support apparatus according to an additional example embodiment of the invention.

FIG. 9 is a perspective view of a support apparatus according to an additional example embodiment of the invention.

FIG. 10 is a perspective view of a support apparatus according to an additional example embodiment of the invention.

FIGS. 11A-11B are schematic illustrations of a portion of the apparatus in different conditions.

FIGS. 12A-12B are schematic illustrations of a portion of the apparatus in different conditions.

FIGS. 13A-13B are schematic illustrations of a portion of the apparatus in different conditions.

### DESCRIPTION

The present invention relates to surgical procedures that employ the use of patient mounted structures for the purpose of providing surgical tool support and/or assisting in image-based guidance. More particularly, the present invention relates to an apparatus comprising a support structure, for supporting a medical device on a patient, that incorporates mounting pads that operate on principles of frictional locking or jamming elements to secure the support structure to the patient.

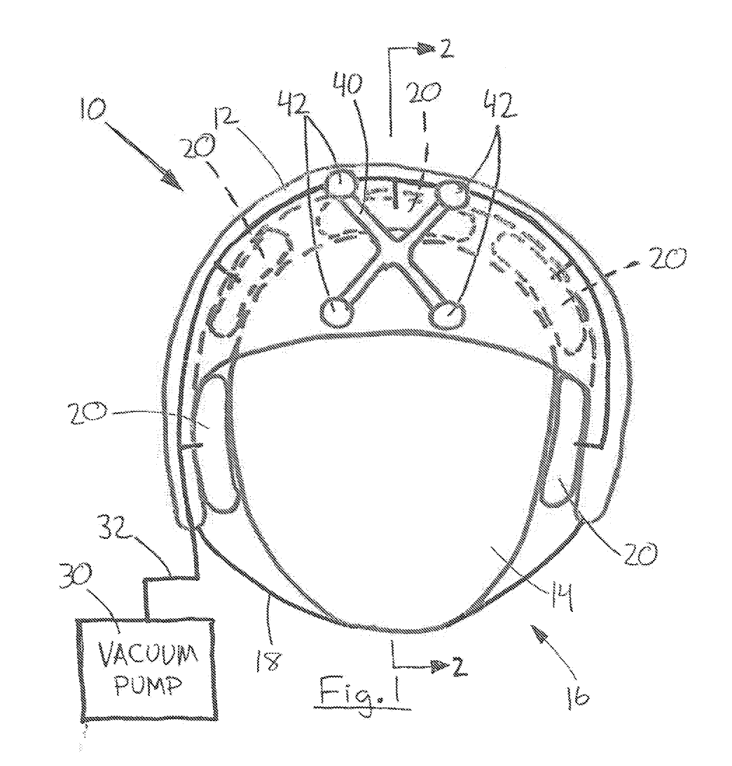

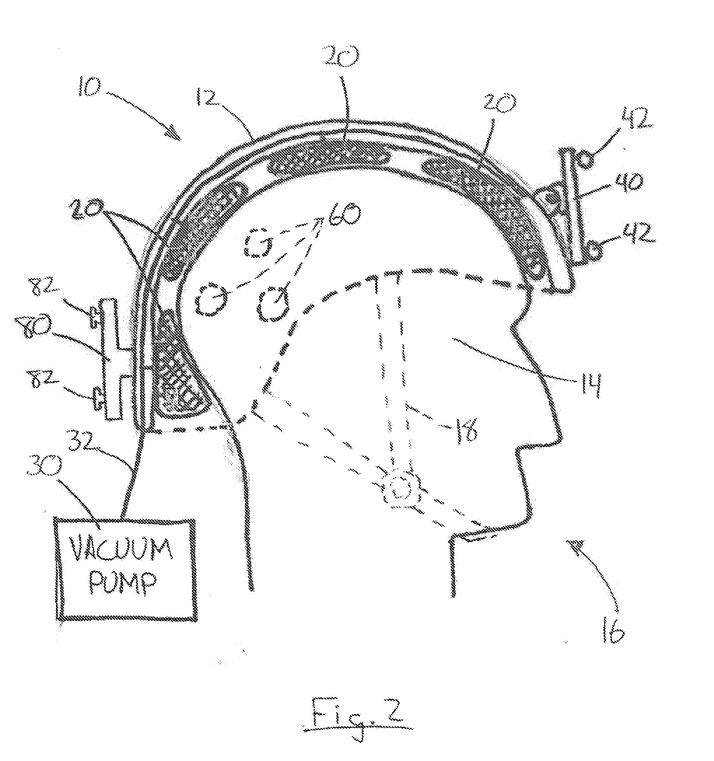

The present invention relates to an apparatus comprising a support structure adapted to be mounted on the anatomy of a patient. The apparatus can, for example, be used to support on the patient a medical device, such as a surgical tool or a rigid body comprising a target fiducial for use in image guided surgical procedures. Referring to FIGS. 1-2, according to one example, the apparatus 10 includes a support structure 12 for being fitted onto the head 14 of a patient 16. In the example embodiment of FIGS. 1-2, the illustrated support structure 12 can resemble or be constructed in the general form of a helmet.

The apparatus 10 includes a plurality of mounting pads 20 that are connected to the support structure 12. The mounting pads 20 are configured and arranged to engage the patient's head 14. A flexible elongated retaining member, such as a strap 18, helps to secure the support structure 12 to the patient 16. The apparatus 10 further includes a vacuum source 30, such as a vacuum pump, with vacuum lines 32 that are fed into the support structure 12 and connected to each of the mounting pads 20.

In the embodiment of FIGS. 1-2, the apparatus 10 includes a medical device in the form of a rigid body 40 that supports a plurality of target fiducials 42. The rigid body 40 is connected to the support structure 12. The target fiducials 42 are configured for use in target registration for image guided surgery. The configuration, arrangement, and location of the rigid body 40 in FIGS. 1-2 is for purposes of illustration and is not meant to limit or restrict the configuration of the apparatus 10. Those skilled in the art will appreciate that the rigid body can have various configurations and arrangements without departing from the spirit and scope of the present invention.

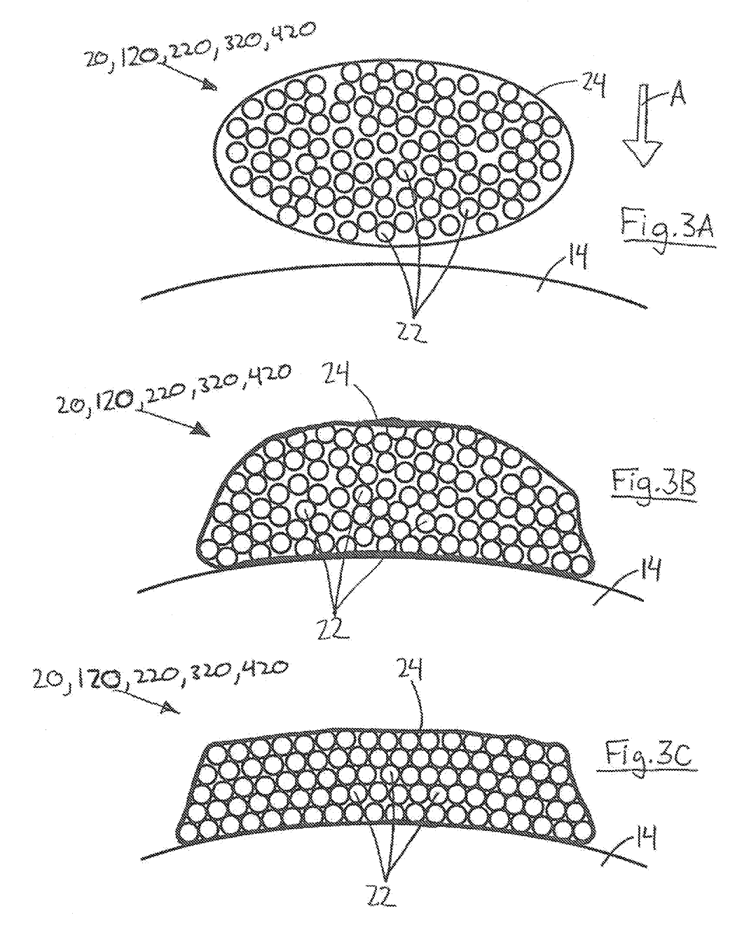

According to one aspect of the invention, the mounting pads 20 can be granular jamming mounting pads. Referring to FIGS. 3A-3C, the mounting pads 20 include a granular material 22 enclosed in a container or confinement referred to herein as a casing 24. The casing 24 is constructed of an airtight or substantially airtight material that is generally deformable and elastic, such as a plastic, polymer, or rubber material. In one example, the casing 24 can be constructed of latex rubber. Alternative materials could be used. The material used to construct the casing 21 can exhibit a high coefficient of friction with human skin.

The granular material 22 is a generally rigid, low density material that tends to move or flow easily under force into unoccupied spaces. Example granular materials 22 include silica granules, plastic or polymer beads, and even coffee grounds. It has been found that using coffee grounds as the granular material 22 can provide a good combination of weight, density, and the propensity to flow easily.

Referring to FIG. 3A with reference to FIGS. 1-2, to use the apparatus 10, the support structure 12 is positioned over the patient's head 14 and brought toward the head, as indicated generally by the arrow A in FIG. 3A. The support structure 12 eventually reaches the head 14 and the support structure is pressed into place, with the strap 18 being used to maintain its position.

Referring to FIG. 3B, as the support structure 12 is pressed onto the patient's head 14, the mounting pads 20 become compressed between the support structure 12 and the patient's head 14. The pressure applied to the support structure 12 causes the casing 24 to deform and the granular material 22 to flow into the spaces created by this deformation. The compression force also causes the granular material 22 to flow into and fill gaps, voids, and other spaces between the grains. In doing so, the mounting pads 20 begin to conform to the shape of the patient's head 14.

At this point, with the support structure 12 pressed onto the patient's head 14 and secured with the strap 18, the vacuum pump 30 is activated. The pump removes air from within the casing 24, creating a vacuum in the mounting pads 20. Referring to FIG. 3C, the vacuum causes the casing 24 to be drawn-in and further compact the granular material 22. The elastic properties of the casing 24 cause it to conform closely to the assumed shape of the volume of granular material 22. This further compaction causes the mounting pads 20 to harden and become rigid. Because the mounting pads 20 were first compressed onto the patient's head 14, the mounting pads, when under vacuum, assume a hardened rigid form that matches the contour of the patient's head 14. The mounting pads 20 thus provide a close fit that is custom tailored to the specific anatomy of the patient 16.

Referring to FIGS. 1-2, the mounting pads 20 provide the close fit described above at locations spaced about the support structure 12. Due to the configuration of the support structure 12 and the spacing and arrangement of the mounting pads 20, the support structure and pads extend around and support the patient's head 14 in a cup-like manner in which some of the pads extend beyond the widest portion of the head and converge toward each other. Put another way, the support structure 12 spaces the mounting pads 20 about the patient's head 14 in a manner similar to the arrangement of a ball-and-socket joint in which the socket extends beyond the equator of the ball. The mounting pads 20, extending around the head 14 in this manner, being drawn into engagement with the head via the strap 18 with the vacuum applied to render the pads in a rigid form-fitting condition, secures the support structure 12 to the head in a stable and secure manner.

Configured in this manner, the support structure 12 is highly resistant to movement relative to the patient's head during surgical procedures. The apparatus 10 thus can provide a rigid and reliable support for the rigid body 40 and the tracking fiducials 42. The apparatus 10 can thus exhibit resistance to target registration errors (TREs) that are commensurate with, or even lower than, those associated with bone screw mounted rigid bodies.

As an additional feature, the support structure 12 can include features for helping to maintain the position of the patient's head 14 during the procedure. As shown in FIG. 2, the support structure 12 can include a bracket 80 that includes fastening structures 82 that cooperate with a patient support structure (not shown), such as an operating table, in order to stabilize the patients head in order to prevent unwanted movements during the surgical procedure. The fastening structures 82 can have any configuration suited to facilitate the connection with the patient support structure. In the embodiment illustrated in FIG. 2, the fastening structures are T-shaped pins that can cooperate with mating T-shaped slots in the patient support structure to help secure that patient 16 to the structure. Alternative fastening structures, such as threaded fasteners, could be used. Additionally, the bracket 80 could be located at any suitable position on the support structure 12.

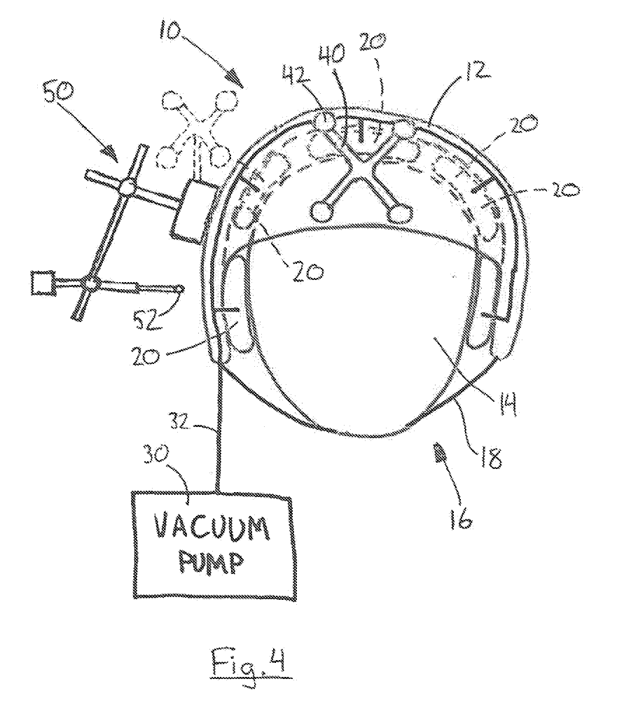

The apparatus 10 can be adapted to support on the patient's head 14 objects other than, or in addition to, the rigid body 40. For example, the apparatus 10 can be adapted to support surgical tools for mounting on the patient 16. Referring to FIG. 4, the apparatus 10 is adapted to support a medical device in the form of a surgical tool 50. The surgical tool can be any device or instrument that may be required to perform a surgical task. For instance, the tool 50 can be an endoscope for viewing a surgical field, a drill for accessing a target site in the anatomy, or a probe having tool ends, such as forceps, graspers, cutters, needles, drug delivery devices, or ablation elements. The tool 50 can be manually or robotically operated, and can be adapted to perform any type of surgical operation where such tools may be useful, such as an endonasal surgical procedure. The apparatus 10 shown in FIG. 4 is identical to that described above with respect to FIGS. 1-3C. In fact, as shown in FIG. 2, the support structure 12 can include mounts 60, such as threaded openings, for receiving fasteners for mounting the surgical tool 50 to the support structure.

In the embodiment of FIG. 4, the attachment point for the tool 50 can be repeatable and known relative to the fiducials 42 on the rigid body 40. Because of this, advantageously, the tracking fiducials 42 on the rigid body 40 can be used to register both the patient's head 14 and the tip 52 of the surgical tool 50. This eliminates the need to separately mount the rigid body 40 and therefore eliminates one potential point where registration errors could occur. In another configuration, the surgical tool 50 could include the tracking fiducials as an integral portion of the surgical tool 50. This is shown in phantom in FIG. 4. In this configuration, the relative positions of the fiducials and the tool base would be fixed. The position of the tool arm and the position of the patients head could be registered using this integrated tool/fiducial configuration.

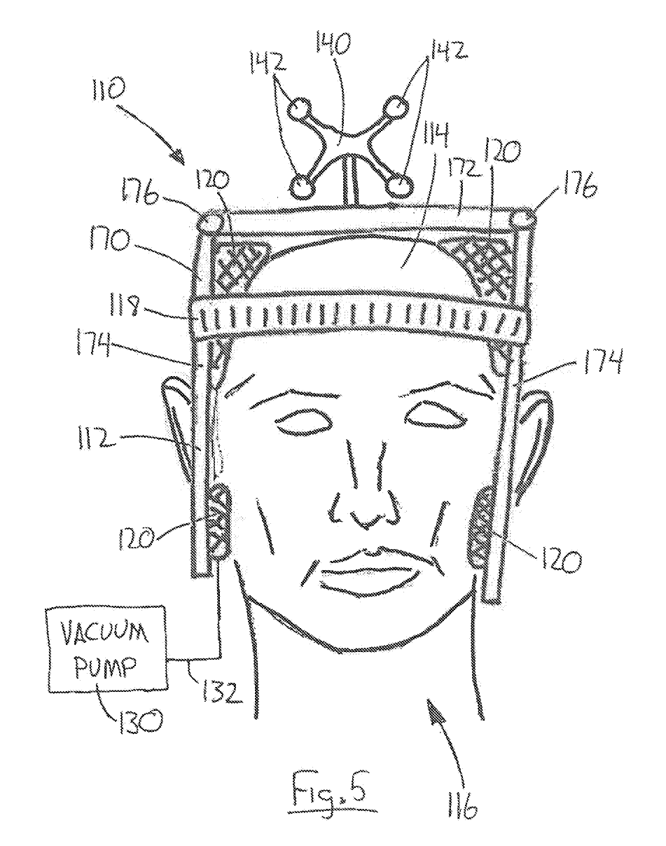

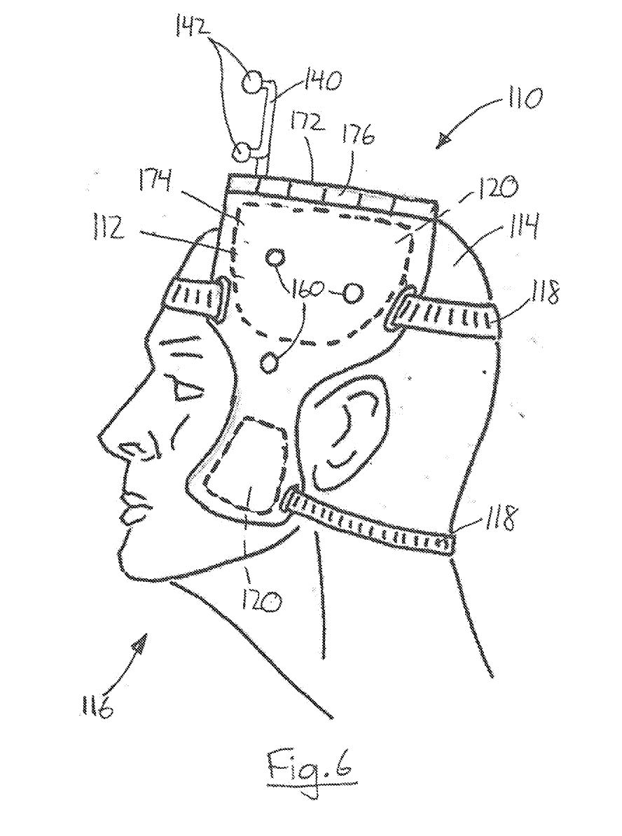

Another example embodiment of the invention is illustrated in FIGS. 5-6. The apparatus 110 of FIGS. 5-6 is similar to the apparatus 10 of FIGS. 1-4, as described above. In the example embodiment of FIGS. 5-6, the apparatus 110 includes a support structure 112 for being fitted onto the head 114 of a patient 116. In the example embodiment of FIGS. 5-6, the support structure 112 is in the form of an articulated frame 170.

The frame 170 includes an upper cross member 172 and a pair of side plates 174 connected to opposite ends of the cross member 172 via a hinged connections 176. The cross member 172 can have a width that is adjustable to accommodate different patient head sizes. A plurality of mounting pads 120 are connected to the support structure 112, specifically to the side plates 174. The mounting pads 120 are configured and arranged to engage the patient's head 114. A pair of flexible elongated retaining members, such as straps 118, help to secure the support structure 112 to the patient 116. The apparatus 110 further includes a vacuum source 130, such as a vacuum pump, with vacuum lines 132 that are fed into the support structure 112 and connected to each of the mounting pads 120.

In the embodiment of FIGS. 5-6, the apparatus 110 includes a medical device in the form of a rigid body 140 that supports a plurality of target fiducials 142. The rigid body 140 is connected to the support structure 112, specifically to the cross member 172. The target fiducials 142 are configured for use in target registration for image guided surgery. The configuration, arrangement, and location of the rigid body 140 in FIGS. 5-6 is for purposes of illustration and is not meant to limit or restrict the configuration of the apparatus 110. Those skilled in the art will appreciate that the rigid body can have various configurations and arrangements without departing from the spirit and scope of the present invention.

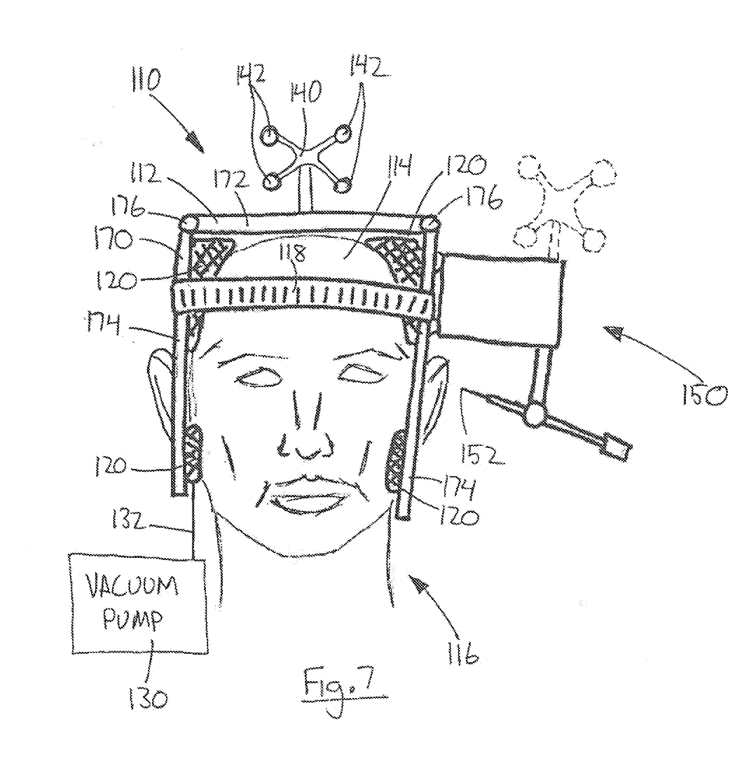

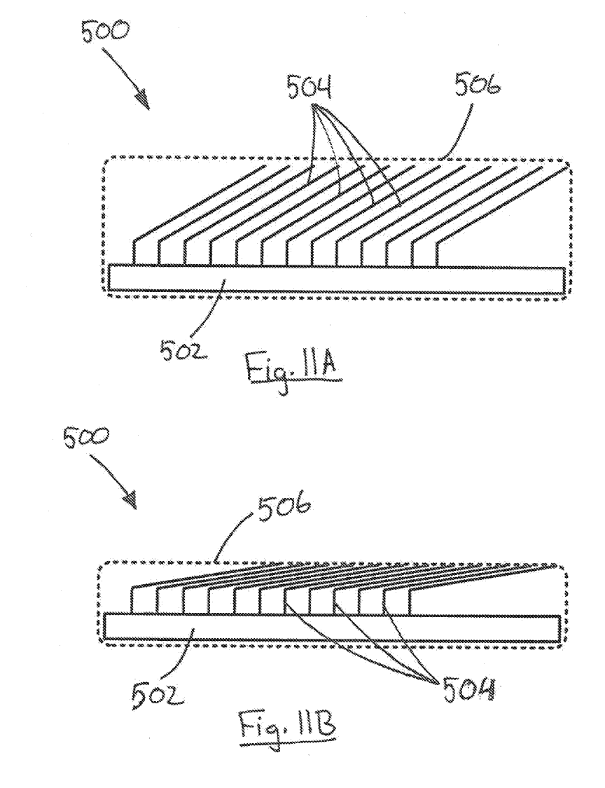

According to the invention, the mounting pads 120 can be granular jamming mounting pads that are constructed and operate in accordance to the description set forth above with reference to FIGS. 3A-3C, or frictional sheet locking or jamming mounting pads as set forth below with reference to FIGS. 11A-13B. Referring to FIGS. 6-7 with reference to FIGS. 3A-3C, to use the apparatus 110, the support structure 112 is positioned over the patient's head 114 and brought toward the head, as indicated generally by the arrow A in FIG. 3A. The support structure 112 eventually reaches the head 14 and the support structure is pressed or held firmly in place while the straps 118 are tightened.

As the straps 118 are tightened, the side plates 174 pivot relative to the cross member 172 and clamp onto the patient's head 114. The mounting pads 120 become compressed between the support structure 112 and the patient's head 114. The pressure applied to the support structure 112 causes the casing 24 of the mounting pads 120 to deform and the granular material 22 to flow into the spaces created by this deformation. The compression force also causes the granular material 22 to flow into and fill gaps, voids, and other spaces between the grains. In doing so, the mounting pads 120 begin to conform to the shape of the patient's head 114.

At this point, with the support structure 112 pressed firmly onto the patient's head 114 and secured with the straps 118, the vacuum pump 130 is activated. The pump 130 removes air from within the casing 24 of the mounting pads 120, creating a vacuum in the pads. Referring to FIG. 3C, the vacuum causes the casing 24 to be drawn-in and further compact the granular material 22. The elastic properties of the casing 24 cause it to conform closely to the assumed shape of the volume of granular material 22. This further compaction causes the mounting pads 120 to harden and become rigid. Because the mounting pads 120 were first compressed onto the patient's head 114, the mounting pads, when under vacuum, assume a hardened rigid form that matches the contour of the patient's head 114. The mounting pads 120 thus provide a close fit that is custom tailored to the specific anatomy of the patient 116.

Referring to FIGS. 5-6, the mounting pads 120 provide the close fit described above at locations spaced about the support structure 112. Due to the configuration of the support structure 112 and the spacing and arrangement of the mounting pads 120, the support structure and pads extend around and support the patient's head 114 in a manner in which some of the pads extend beyond the widest portion of the head and converge toward each other. As best shown in FIG. 5, the uppermost mounting pads 120 extend partially around the top of the patient's head 114 and the lowermost pads extend inward beneath the area of the cheek bone. This creates an interference fit in which the mounting pads 120, extending around the head 114 in this manner and being drawn into engagement with the head via the straps 118 with the vacuum applied to render the pads in a rigid form-fitting condition, secures the support structure 112 to the head in a stable and secure manner.

Configured in this manner, the support structure 112 is highly resistant to movement relative to the patient's head during surgical procedures. The apparatus 110 thus can provide a rigid and reliable support for the rigid body 140 and the tracking fiducials 142. The apparatus 112 can thus exhibit resistance to target registration errors (TREs) that can approach, or become commensurate with those associated with bone screw mounted rigid bodies.

The apparatus 110 of FIGS. 5-6 can also be adapted to support on the patient's head 114 objects other than, or in addition to, the rigid body 140. For example, the apparatus 110 can be adapted to support surgical tools for mounting on the patient 116. Referring to FIG. 7, the apparatus 110 is adapted to support a medical device in the form of a surgical tool 150. The surgical tool can be any device or instrument that may be required to perform a surgical task. For instance, the tool 150 can be an endoscope for viewing a surgical field, a drill for accessing a target site in the anatomy, or a probe having tool ends, such as forceps, graspers, cutters, needles, drug delivery devices, or ablation elements. The tool 150 can be manually or robotically operated, and can be adapted to perform any type of surgical operation where such tools may be useful, such as an endonasal surgical procedure. The apparatus 110 shown in FIG. 7 is identical to that described above with respect to FIGS. 5-6 and 3A-3C. In fact, as shown in FIG. 6, the support structure 112 can include mounts 160, such as threaded openings, for receiving fasteners for mounting the surgical tool 150 to the support structure.

In the embodiment of FIG. 7, the attachment point for the tool 150 can be repeatable and known relative to the fiducials 142 on the rigid body 140. Because of this, advantageously, the tracking fiducials 142 on the rigid body 140 can be used to register both the patient's head 114 and the tip 152 of the surgical tool 150. This eliminates the need to separately mount the rigid body 140 and therefore eliminates one potential point where registration errors could occur. In another configuration, the surgical tool 150 could include the tracking fiducials as an integral portion of the surgical tool 150. This is shown in phantom in FIG. 7. In this configuration, the relative positions of the fiducials and the tool base would be fixed. The position of the tool arm and the position of the patients head could be registered using this integrated tool/fiducial configuration.

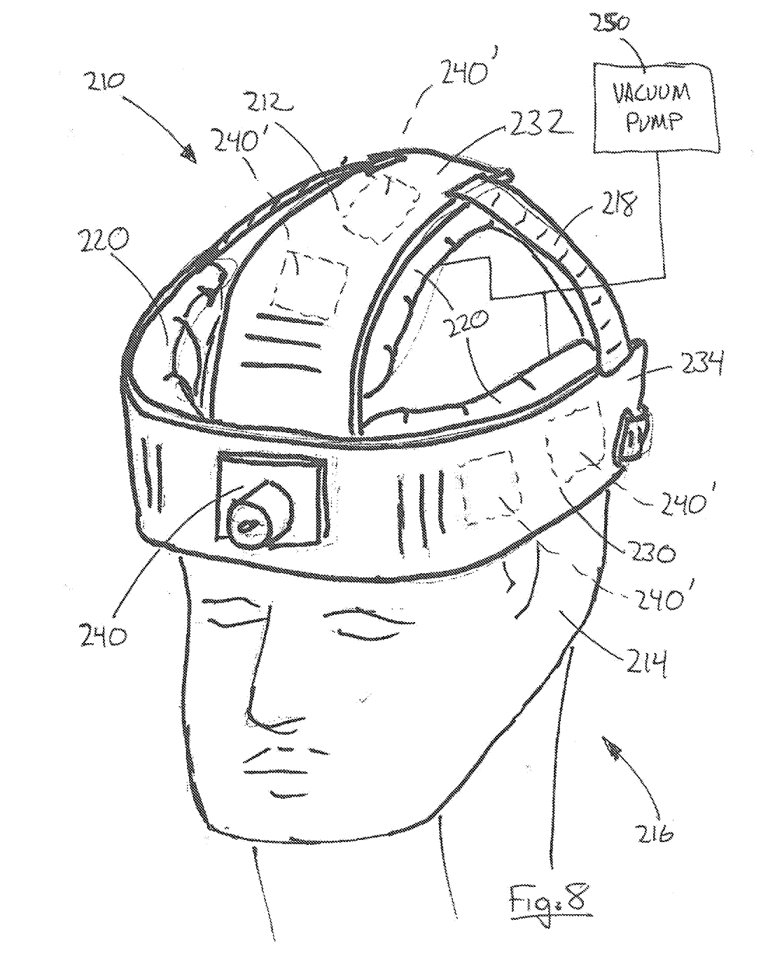

Another example embodiment of the invention is illustrated in FIG. 8. The apparatus 210 of FIG. 8 is similar to those of FIGS. 1-7, as described above. In the example embodiment of FIG. 8, the apparatus 210 includes a support structure 212 for being fitted onto the head 214 of a patient 216. In the example embodiment of FIG. 8, the support structure 212 includes C-shaped frame 230 that extends laterally around the patient's head 214, across the forehead and wrapping at least partially around the back of the head. A top member 232 is connected to the middle of the frame 230 and extends arcuately up and over the top of the patients head 214. The ends 234 of the C-shaped frame 230 and the free end of the top member 232 are interconnected by a flexible elongated member 218, such as a strap that can be tightened/loosened to secure the support structure 212 on the patient's head 214. This tightening/loosening can be achieved, for example, via a buckle or an adjusting wheel, such as those commonly found on safety helmets. The lengths of the C-shaped frame 230 and the top member 232 can be adjustable to accommodate heads of different sizes and shapes.

The apparatus 210 of FIG. 8 includes at least one device mount 240 connected to either the frame 230 or top member 212. The device mount 240 is illustrated in center position on the frame 230. Examples of additional or alternative locations for tool holders on both the frame 230 and top member 232 are illustrated in dashed lines at 240′. The device mount 240 can be adapted to hold any of the medical devices disclosed and described herein, such as such as a rigid body supporting target fiducials, a surgical tool, a patient support bracket, or any combination of these devices. The device mount 240 may include a quick release mechanism to facilitate swapping medical devices during a procedure.

The frame 230 can be configured to envelope a portion of the head that allows the ends of the C-shaped structure to converge toward each other so that the structure. i.e., the spacing between the ends 234 is smaller than the diameter of the patient's head. In this configuration, the frame 230 can be constructed to be generally stiff and rigid, and yet at least somewhat elastic to allow the ends 234 to move apart in order to fit the structure over the patient's head 214. The straps 218 can help to further secure the support structure 212 to the patient 216.

The frame 230 and top member support one or more mounting pads 220. The mounting pads 220 are configured and arranged to engage the patient's head 214. According to the invention, the mounting pads 220 can be granular jamming mounting pads that are constructed and operate in accordance to the description set forth above with reference to FIGS. 3A-3C, or frictional sheet locking or jamming mounting pads as set forth below with reference to FIGS. 11A-13B. Referring to FIG. 8 with reference to FIGS. 3A-3C, to use the apparatus 210, the support structure 212 is positioned over the patient's head 214 and brought toward the head, as indicated generally by the arrow A in FIG. 3A. The support structure 212 eventually reaches the head 214 and the support structure is pressed or held firmly in place due to the elastic construction of the frame 230 and top member 232. The straps 218 can help maintain the position of the support structure 212 on the patient 216.

The mounting pads 220 become compressed between the support structure 212 and the patient's head 214. The pressure applied to the support structure 212 causes the casing 24 of the mounting pads 220 to deform and the granular material 22 to flow into the spaces created by this deformation. The compression force also causes the granular material 22 to flow into and fill gaps, voids, and other spaces between the grains. In doing so, the mounting pads 220 begin to conform to the shape of the patient's head 214.

At this point, with the support structure 212 pressed firmly onto the patient's head 214 and secured with the straps 218, the vacuum pump 250 is activated. The vacuum pump 250 removes air from within the casing 224 of the mounting pads 220, creating a vacuum in the pads. Referring to FIG. 3C, the vacuum causes the casing 24 to be drawn-in and further compact the granular material 22. The elastic properties of the casing 24 cause it to conform closely to the assumed shape of the volume of granular material 22. This further compaction causes the mounting pads 220 to harden and become rigid. Because the mounting pads 220 were first compressed onto the patient's head 214, the mounting pads, when under vacuum, assume a hardened rigid form that matches the contour of the patient's head 214. The mounting pads 220 thus provide a close fit that is custom tailored to the specific anatomy of the patient 210.

Referring to FIG. 8, the mounting pads 220 provide the close fit described above at locations spaced about the support structure 212. Due to the configuration of the support structure 212 and the spacing and arrangement of the mounting pads 220, the support structure and pads extend around and support the patient's head 214 in a manner in which some of the pads extend beyond the widest portion of the head and converge toward each other. The mounting pads 220 extend around the sides and top of the patient's head 214 and create an interference fit that maintains the support structure 212 in place on the head. The mounting pads 220, extending around the head 214 in this manner and being drawn into engagement with the head via the straps 218 with the vacuum applied to render the pads in a rigid form-fitting condition, secures the support structure 212 to the head in a stable and secure manner.

Configured in this manner, the support structure 212 is highly resistant to movement relative to the patient's head during surgical procedures. The apparatus 210 thus can provide a rigid and reliable support for the medical device(s) attached to the device mount(s) 240. Where the medical device attached to the device mount 240 includes a target fiducial, the apparatus 212 provides resistance to target registration errors (TREs) to a degree that can approach, or become commensurate with those associated with bone screw mounted rigid bodies.

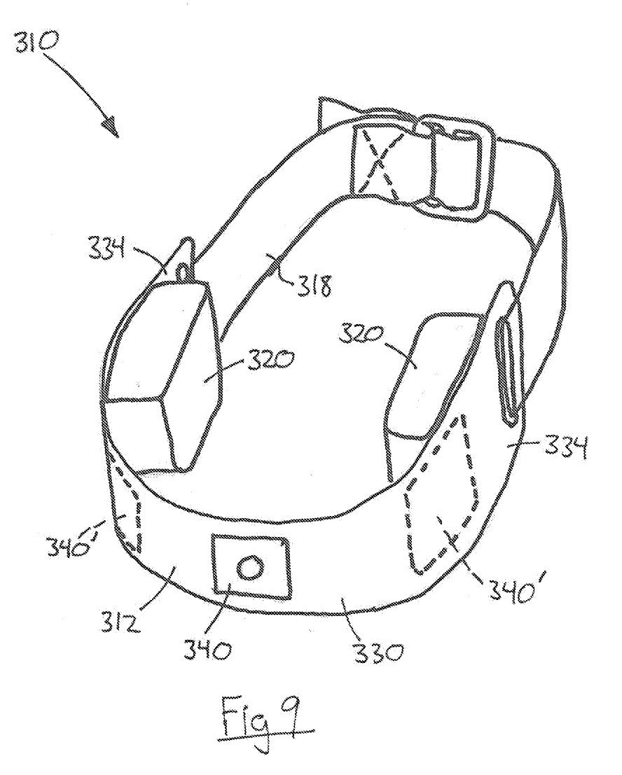

Another example embodiment of the invention is illustrated in FIG. 9. The apparatus 310 of FIG. 9 is similar to those of FIGS. 1-8, as described above. In the example embodiment of FIG. 9, the apparatus 310 includes a support structure 312 for being fitted onto the head of a patient (not shown). In the example embodiment of FIG. 9, the support structure 312 includes a generally C-shaped frame 330 in the form of a curved flat plate formed of a semi-rigid yet flexible material, e.g., a plastic or polymer such as polyethylene. The frame 330 is sized, proportioned and otherwise configured to wrap partially around the patient's head, across the forehead and along the sides of the head generally over the temples and above the ears. The ends 334 of the C-shaped frame 330 are interconnected by a flexible elongated member 318, such as a strap that can be tightened/loosened to secure the support structure 312 on the patient's head. This tightening/loosening can be achieved, for example, via a buckle or an adjusting wheel, such as those commonly found on safety helmets. The length and configuration of the C-shaped frame 330 can be adjustable to accommodate heads of different sizes and shapes and also to accommodate portions of the patient's anatomy.

The apparatus 310 of FIG. 9 includes at least one device mount 340 connected to the frame 330. The device mount 340 is illustrated in center position on the frame 330. Examples of additional or alternative locations for tool holders on the frame 330 are illustrated in dashed lines at 340′. The device mount 340 can be adapted to hold any of the medical devices disclosed and described herein, such as such as a rigid body supporting target fiducials, a surgical tool, a patient support bracket, or any combination of these devices. The device mount 340 may include a quick release mechanism to facilitate swapping medical devices during a procedure.

The frame 330 can be configured to envelope any desired circumferential portion of the head. For example, the frame 330 can be configured to allow the ends 334 of the C-shaped structure to converge toward each other so that the structure. i.e., the spacing between the ends is smaller than the diameter of the patient's head. In this configuration, the semi-rigid frame 330 can be constructed to allow the ends 334 to move apart in order to fit the structure over the patient's head. The straps 318 can help to further secure the support structure 312 to the patient.

The frame 330 supports one or more mounting pads 320 configured and arranged to engage the patient's head. In the example illustrated in FIG. 9, the mounting pads 320 are fixed to the frame 330 at locations adjacent or near the opposite ends 334. In the example illustrated in FIG. 9, the mounting pads 320 are granular jamming mounting pads that are constructed and operate in accordance to the description set forth above with reference to FIGS. 3A-3C, or frictional sheet locking or jamming mounting pads as set forth below with reference to FIGS. 11A-13B. Referring to FIG. 9 with reference to FIGS. 3A-3C, to use the apparatus 310, the support structure 312 is positioned over the patient's head and brought toward the head, as indicated generally by the arrow A in FIG. 3A. The support structure 312 eventually reaches the head and the support structure is pressed or held firmly in place due to the elastic construction of the frame 330. The straps 318 can help maintain the position of the support structure 312 on the patient.

The mounting pads 320 become compressed between the support structure 312 and the patient's head. The pressure applied to the support structure 312 causes the casing 24 of the mounting pads 320 to deform and the granular material 22 to flow into the spaces created by this deformation. The compression force also causes the granular material 22 to flow into and fill gaps, voids, and other spaces between the grains. In doing so, the mounting pads 320 begin to conform to the shape of the patient's head.

At this point, with the support structure 312 pressed firmly onto the patient's head and secured with the straps 318, the vacuum pump (not shown) is activated. The vacuum pump removes air from within the casing 324 of the mounting pads 320, creating a vacuum in the pads. Referring to FIG. 3C, the vacuum causes the casing 24 to be drawn-in and further compact the granular material 22. The elastic properties of the casing 24 cause it to conform closely to the assumed shape of the volume of granular material 22. This further compaction causes the mounting pads 320 to harden and become rigid. Because the mounting pads 320 were first compressed onto the patient's head, the mounting pads, when under vacuum, assume a hardened rigid form that matches the contour of the patient's head. The mounting pads 320 thus provide a close fit that is custom tailored to the specific anatomy of the patient.

Referring to FIG. 9, the mounting pads 320 provide the close fit described above at locations spaced about the support structure 312. Due to the configuration of the support structure 312 and the spacing and arrangement of the mounting pads 320, the support structure and pads extend around and support the patient's head in a manner in which some of the pads extend beyond the widest portion of the head and converge toward each other. The mounting pads 320 extend around the sides of the patient's head and create an interference fit that maintains the support structure 312 in place on the head. The mounting pads 320, extending around the head in this manner and being drawn into engagement with the head via the straps 318 with the vacuum applied to render the pads in a rigid form-fitting condition, secures the support structure 312 to the head in a stable and secure manner.

Configured in this manner, the support structure 312 is highly resistant to movement relative to the patient's head during surgical procedures. The apparatus 310 thus can provide a rigid and reliable support for the medical device(s) attached to the device mount(s) 340. Where the medical device attached to the device mount 340 includes a target fiducial, the apparatus 310 provides resistance to target registration errors (TREs) to a degree that can approach, or become commensurate with those associated with bone screw mounted rigid bodies.

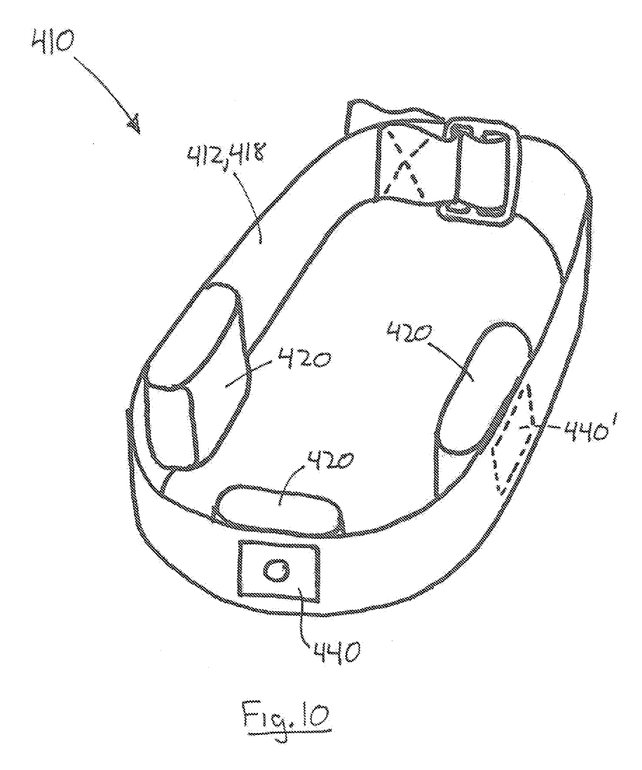

Another example embodiment of the invention is illustrated in FIG. 10. The apparatus 410 of FIG. 10 is similar to those of FIGS. 1-9, as described above. The apparatus 410 of FIG. 10 is especially similar to the apparatus 310 of FIG. 9. In the embodiment of FIG. 10, the support structure 412 is a flexible elongated member 418, such as a strap that extends around the entire circumference of the patient's head and can be tightened/loosened to secure the apparatus 410 to the patients head. This tightening/loosening can be achieved, for example, via a buckle or an adjusting wheel, such as those commonly found on safety helmets.

The strap 418 supports one or more mounting pads 420 configured and arranged to engage the patient's head. In the example illustrated in FIG. 10, the mounting pads 420 are fixed to the strap 418 at three locations: one on each side of the patient's head and one centered on the patient's forehead. The position, location, number, and extent of the mounting pads 420 can be altered, adjusted, or otherwise changed without departing from or otherwise affecting the invention. In the example illustrated in FIG. 10, the mounting pads 420 are granular jamming mounting pads that are constructed and operate in accordance to the description set forth above with reference to FIGS. 3A-3C, or frictional sheet locking or jamming mounting pads as set forth below with reference to FIGS. 11A-13B. Referring to FIG. 10 with reference to FIGS. 3A-3C, to use the apparatus 410, the strap 418 is positioned or wrapped around the patient's head and brought toward the head, as indicated generally by the arrow A in FIG. 3A. The strap 418 and the mounting pads 420 eventually reach the head and the pads are pressed or held firmly in place by tightening the straps 418 to help maintain the position of the support structure 412 on the patient.

The mounting pads 420 become compressed between the strap 418 and the patient's head. The pressure applied to the strap 418 causes the casing 24 of the mounting pads 420 to deform and the granular material 22 to flow into the spaces created by this deformation. The compression force also causes the granular material 22 to flow into and fill gaps, voids, and other spaces between the grains. In doing so, the mounting pads 420 begin to conform to the shape of the patient's head.

At this point, with the mounting pads 420 pressed firmly onto the patient's head and secured with the straps 418, the vacuum pump (not shown) is activated. The vacuum pump removes air from within the casing 24 of the mounting pads 420, creating a vacuum in the pads. Referring to FIG. 3C, the vacuum causes the casing 24 to be drawn-in and further compact the granular material 22. The elastic properties of the casing 24 cause it to conform closely to the assumed shape of the volume of granular material 22. This further compaction causes the mounting pads 420 to harden and become rigid. Because the mounting pads 420 were first compressed onto the patient's head, the mounting pads, when under vacuum, assume a hardened rigid form that matches the contour of the patient's head. The mounting pads 420 thus provide a close fit that is custom tailored to the specific anatomy of the patient.

Referring to FIG. 10, the mounting pads 420 provide the close fit described above at locations spaced along the length of the strap 418. The strap 418 extends around the entire circumference of the patient's head and, in combination with the mounting pads 420, create a fit that maintains the apparatus 410 in a fixed position on the head. The mounting pads 420, extending around the head in this manner and being drawn into engagement with the head via the straps 418 with the vacuum applied to render the pads in a rigid form-fitting condition that secures the apparatus 410 to the head in a stable and secure manner.

In the example embodiment of FIG. 10, the apparatus 410 of includes at least one device mount 440 that is connected directly to one of the mounting pads 420. This can eliminate the need for frames, helmets, or other structures for supporting the device mount 440. The device mount 440 is illustrated on the center mounting pad 420. Examples of additional or alternative locations for device mounts are the other mounting pads or even on the strap 418 itself. The device mount 440 can be adapted to hold any of the medical devices disclosed and described herein, such as such as a rigid body supporting target fiducials, a surgical tool, a patient support bracket, or any combination of these devices. The device mount 440 may include a quick release mechanism to facilitate swapping medical devices during a procedure.

Configured in this manner, the support structure 420 is highly resistant to movement relative to the patient's head during surgical procedures. The apparatus 410 thus can provide a rigid and reliable support fir the medical device(s) attached to the device mount(s) 440. Where the medical device attached to the device mount 440 includes a target fiducial, the apparatus 410 provides resistance to target registration errors (TREs) to a degree that can approach, or become commensurate with those associated with bone screw mounted rigid bodies.

The mounting pads can have configurations other than the granular jamming configurations illustrated in FIGS. 3A-3C. Examples of these alternative mounting pads are illustrated in FIGS. 11A-13B. The mounting pads in FIGS. 11A-13B still operate on principles of jamming under a vacuum, i.e., providing a conforming structure for conforming to the patient's anatomy (e.g., head) during fitting and that once fitted, can be actuated via vacuum to assume a rigid structure due to engagement and friction between the shape conforming elements of the pads in order to provide a stable patient mount. The mounting pads illustrated in the example embodiments of FIGS. 11A-13B are similar to the pads of the embodiments of FIGS. 3A-3C, replacing the granular material used as jamming elements in with elements having a different structure. The principal of operation for the mounting pads remains essentially the same or similar, as described below. The mounting pads of FIGS. 11A-13B can be implemented in any of the example embodiments of the invention described herein.

The mounting pads of FIGS. 11A-13B implement planar sheets of material (e.g., paper or thin plastic) that replace the granular jamming material within the casing. When pressed against the patient, the sheets conform to the contour of the patient's anatomy and are pressed against one another. Upon drawing a vacuum in the casing, the sheets are further urged against one another and lock due to friction between the sheets. This friction lock maintains the assumed shape of the mounting pads, allowing the pads to help support the medical device(s) supported by the apparatus.

Referring to FIGS. 11A-11B, the mounting pad 500 includes a flexible or semi-rigid base or substrate 502 and a plurality of sheets 504 that are connected to the base and extend upward from the base. A casing 506 (shown schematically in dashed lines) encloses the base 502 and sheets 504. The illustration of FIGS. 11A-11B is schematic in nature and illustrated only a few sheets 504, whereas, in application, the mounting pad 500 could include many more sheets, even on the order of several hundred or more. Also, in the embodiment illustrated in FIGS. 11A-11B, the sheets 504 extend upward at an angle. This is by way of example only. The sheets 504 could extend at any angle, including perpendicular to the base.

In this form, the sheets 504 take the form of a series of “leaves” that rise from the flexible substrate 502 in an adjacent, parallel, and overlying manner. When the mounting pad 500 is pressed against the patient, the sheets 504 deflect and conform to the contour of the patient's anatomy. The degree of deflection and the resistance thereto can be configured through selection of the material (e.g., type and/or thickness) used to construct the sheets 504. As shown in FIG. 11B, when a vacuum is drawn in the casing 506, the casing compresses against the sheets 5041 and the sheets are pressed against one another, which locks the mounting pad 504 in the conformed shape due frictional locking between the sheets. Although the compression of the sheets 504 illustrated in FIGS. 11A-11B is generally flat and uniform, it will be appreciated that the sheets can conform to virtually any shape, regular or irregular, and that shape will be locked and maintained when the vacuum is drawn in the casing 506.

The mounting pads 500 of FIGS. 11A-11B can be implemented in any of the apparatus comprising a support structure for supporting a medical device on a patient described herein. Configured in this manner, the mounting pads 500 can help provide a support structure that is highly resistant to movement relative to the patient, e.g., the patient's head, during surgical procedures. The apparatus outfitted with the mounting pads 500 thus can provide a rigid and reliable support for the medical device(s) attached to the support structure. Where the medical device attached to the support structure includes a target fiducial, the mounting pads 500 help provide the apparatus with resistance to target registration errors (TREs) to a degree that can approach, or become commensurate with those associated with bone screw mounted rigid bodies.

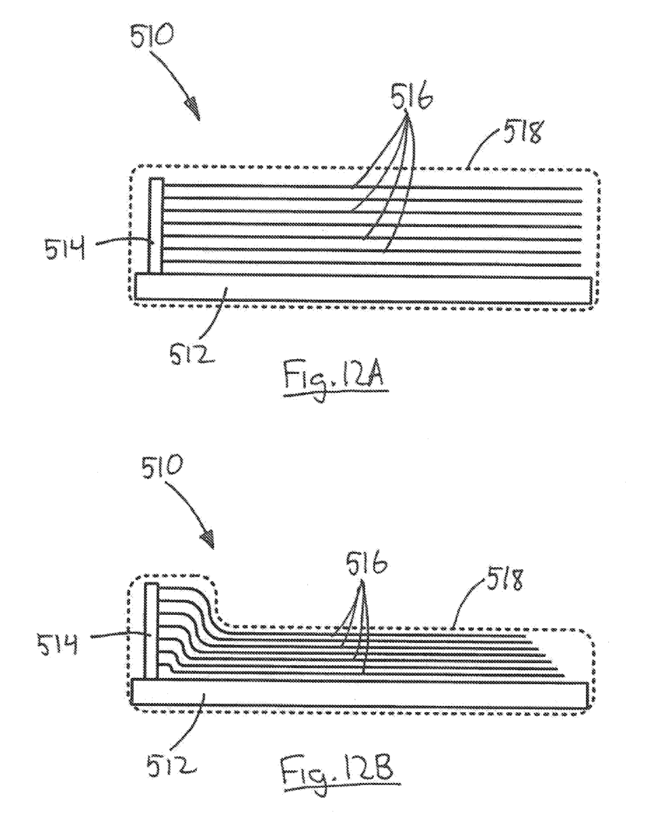

Referring to FIGS. 12A-12B, the mounting pad 510 includes a flexible or semi-rigid base or substrate 512 and a post 514 that extends vertically from the base 512. A plurality of sheets 516 are connected to the post 514 and extend laterally from the post above base 512. A casing 518 (shown schematically in dashed lines) encloses the base 512, post, and sheets 516. The illustration of FIGS. 12A-12B is schematic in nature and illustrated only a few sheets 516, whereas, in application, the mounting pad 510 could include many more sheets, even on the order of several hundred or more. Also, in the embodiment illustrated in FIGS. 12A-12B, the sheets 516 are flat, planar, and extend horizontally above the base 512. This is by way of example only. The sheets 516 could extend at angles from the post 514, and need not be limited to a flat, planar configuration. For instance, the sheets could have a curved or corrugated in construction.

In this form, the sheets 516 take the form of a series of “leaves” that extend above the flexible substrate 512 in an adjacent, parallel, and overlying manner. When the mounting pad 510 is pressed against the patient, the sheets 516 deflect and conform to the contour of the patient's anatomy. The degree of deflection and the resistance thereto can be configured through selection of the material (e.g., type and/or thickness) used to construct the sheets 516. As shown in FIG. 12B, when a vacuum is drawn in the casing 518, the casing compresses against the sheets 516 and the sheets are pressed against one another, which locks the mounting pad 510 in the conformed shape due frictional locking between the sheets. Although the compression of the sheets 516 illustrated in FIGS. 12A-12B is generally flat and uniform, it will be appreciated that the sheets can conform to virtually any shape, regular or irregular, and that shape will be locked and maintained when the vacuum is drawn in the casing 518.

The mounting pads 510 of FIGS. 12A-12B can be implemented in any of the apparatus comprising a support structure for supporting a medical device on a patient described herein. Configured in this manner, the mounting pads 510 can help provide a support structure that is highly resistant to movement relative to the patient, e.g., the patient's head, during surgical procedures. The apparatus outfitted with the mounting pads 510 thus can provide a rigid and reliable support for the medical device(s) attached to the support structure. Where the medical device attached to the support structure includes a target fiducial, the mounting pads 510 help provide the apparatus with resistance to target registration errors (TREs) to a degree that can approach, or become commensurate with those associated with bone screw mounted rigid bodies.

Referring to FIGS. 12A-12B, the mounting pad 510 includes a flexible or semi-rigid base or substrate 512 and a post 514 that extends vertically from the base 512. A plurality of sheets 516 are connected to the post 514 and extend laterally from the post above base 512. A casing 518 (shown schematically in dashed lines) encloses the base 512, post 514, and sheets 516. The illustration of FIGS. 12A-12B is schematic in nature and illustrated only a few sheets 516, whereas, in application, the mounting pad 510 could include many more sheets, even on the order of several hundred or more. Also, in the embodiment illustrated in FIGS. 12A-12B, the sheets 516 are flat, planar, and extend horizontally above the base 512. This is by way of example only. The sheets 516 could extend at angles from the post 514, and need not be limited to a flat, planar configuration. For instance, the sheets could have curved or corrugated constructions.

In this form, the sheets 516 take the form of a series of “leaves” that extend above the flexible substrate 512 in an adjacent, parallel, and overlying manner. When the mounting pad 510 is pressed against the patient, the sheets 516 deflect and conform to the contour of the patient's anatomy. The degree of deflection and the resistance thereto can be configured through selection of the material (e.g., type and/or thickness) used to construct the sheets 516. As shown in FIG. 12B, when a vacuum is drawn in the casing 518, the casing compresses against the sheets 516 and the sheets are pressed against one another, which locks the mounting pad 510 in the conformed shape due frictional locking between the sheets. Although the compression of the sheets 516 illustrated in FIGS. 12A-12B is generally flat and uniform, it will be appreciated that the sheets can conform to virtually any shape, regular or irregular, and that shape will be locked and maintained when the vacuum is drawn in the casing 518.

The mounting pads 510 of FIGS. 12A-12B can be implemented in any of the apparatus comprising a support structure for supporting a medical device on a patient described herein. Configured in this manner, the mounting pads 510 can help provide a support structure that is highly resistant to movement relative to the patient, e.g., the patient's head, during surgical procedures. The apparatus outfitted with the mounting pads 510 thus can provide a rigid and reliable support for the medical device(s) attached to the support structure. Where the medical device attached to the support structure includes a target fiducial, the mounting pads 510 help provide the apparatus with resistance to target registration errors (TREs) to a degree that can approach, or become commensurate with those associated with bone screw mounted rigid bodies.

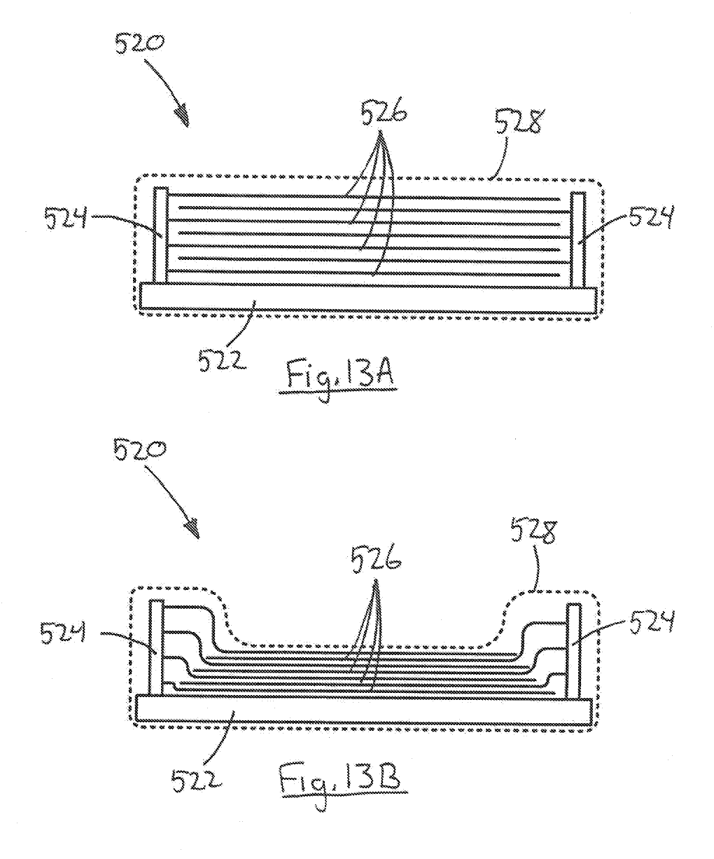

Referring to FIGS. 13A-13B, the mounting pad 520 includes a flexible or semi-rigid base or substrate 522 and posts 524 that extend vertically from the base 522 at opposite ends thereof. A plurality of sheets 526 are connected to the posts 524 and extend laterally from the posts above base 522. The sheets from the opposite posts 524 are interleaved or collated. A casing 528 (shown schematically in dashed lines) encloses the base 522, posts 524, and sheets 520. The illustration of FIGS. 13A-13B is schematic in nature and illustrated only a few sheets 526, whereas, in application, the mounting pad 520 could include many more sheets, even on the order of several hundred or more. Also, in the embodiment illustrated in FIGS. 13A-13B, the sheets 526 are flat, planar, and extend horizontally above the base 522. This is by way of example only. The sheets 526 could extend at angles from the post 524, and need not be limited to a flat, planar configuration. For instance, the sheets could have curved or corrugated constructions.

In this form, the sheets 526 take the form of a series of “leaves” that extend above the flexible substrate 522 in an adjacent, parallel, overlying, and interleaved manner. When the mounting pad 520 is pressed against the patient, the sheets 526 deflect and conform to the contour of the patient's anatomy. The degree of deflection and the resistance thereto can be configured through selection of the material (e.g., type and/or thickness) used to construct the sheets 526. As shown in FIG. 13B, when a vacuum is drawn in the casing 528, the casing compresses against the sheets 526 and the sheets are pressed against one another, which locks the mounting pad 520 in the conformed shape due frictional locking between the sheets. Although the compression of the sheets 526 illustrated in FIGS. 13A-13B is generally flat and uniform, it will be appreciated that the sheets can conform to virtually any shape, regular or irregular, and that shape will be locked and maintained when the vacuum is drawn in the casing 528.

The mounting pads 520 of FIGS. 13A-13B can be implemented in any of the apparatus comprising a support structure for supporting a medical device on a patient described herein. Configured in this manner, the mounting pads 520 can help provide a support structure that is highly resistant to movement relative to the patient, e.g., the patient's head, during surgical procedures. The apparatus outfitted with the mounting pads 520 thus can provide a rigid and reliable support for the medical device(s) attached to the support structure. Where the medical device attached to the support structure includes a target fiducial, the mounting pads 520 help provide the apparatus with resistance to target registration errors (TREs) to a degree that can approach, or become commensurate with those associated with bone screw mounted rigid bodies.

From the above, it will be appreciated that the present invention provides a non-surgically attached stable platform for supporting rigid body fiducial mounts and/or surgical tools during surgery. Although specific locations of these devices are depicted in the figures, the apparatus could be configured to position the devices at any desired location relative to the patient's head.

Additionally, the specific configurations of the support structure is not meant to be limited to those illustrated in the figures. The support structure can have any configuration suitable to: a) apply pressure to the mounting pads so that they conform to the patient's head, and b) provide a stable support for the mounted medical device. Those skilled in the art will appreciate that this functionality can be achieved with a variety of support structure configurations.

While example embodiments have been presented in the foregoing detailed description, it should be appreciated that variations of these embodiments can exist without departing from the spirit and scope of the invention. The embodiments described herein are not intended to limit the scope, applicability, or configuration of the invention. Rather, the detailed description provides sufficient detail to enable one skilled in the art to make and use the invention. Those skilled in the art will perceive applications, improvements, changes and modifications to the invention. Such applications, improvements, changes and modifications within the skill of the art are intended to be covered by the appended claims.

1. An apparatus for supporting a medical device on a patient, comprising:
a support structure adapted to be fitted to the patient's body, the medical device being connectable to the support structure; and
at least one mounting pad connected to the support structure and being adapted to engage the patient's anatomy, the at least one mounting pad comprising a frictional jamming structure configured so that, in a non-actuated condition of the mounting pad, the mounting pad conforms to the patient's anatomy and, in an actuated condition of the mounting pad, the mounting pad becomes rigid in its conformed shape.

2. The apparatus recited in claim 1, further comprising a flexible elongated member, such as a strap, for helping to apply a force to the support structure that compresses the at least one mounting pad between the support structure and the patient.

3. The apparatus recited in claim 1, wherein the at least one mounting pad comprises a casing that at least partially surrounds the jamming structure, the apparatus further comprising a vacuum source operatively connected to the at least one mounting pad and being operative to draw a vacuum in the casing of the at least one mounting pad.

4. The apparatus recited in claim 1, wherein the jamming structure comprises a volume of granular material.

5. The apparatus recited in claim 4, wherein the granular material comprises at least one of granular silica, polymer beads of varying shapes and configurations, and coffee grounds.

6. The apparatus recited in claim 3, wherein the casing is constructed out of at least one of a plastic material, a polymer material, and a natural or synthetic rubber material.

7. The apparatus recited in claim 3, wherein the casing has a latex rubber construction.

8. The apparatus recited in claim 1, wherein the support structure is adapted for connection to the patient's head.

9. The apparatus recited in claim 8, wherein the support structure comprises a shell in the general form of a helmet.

10. The apparatus recited in claim 8, wherein the support structure comprises a frame comprising a cross member having an adjustable width, and side plates connected to opposite ends of the cross member via a hinged connection.

11. The apparatus recited in claim 8, wherein the support structure a semi-rigid plate adapted to be at least partially wrapped around the patient's head, and a strap connected to opposite ends of the plate for securing the plate to the patient's head.

12. The apparatus recited in claim 8, wherein the support structure comprises a strap to which the mounting pads are connected, the strap being adapted to wrap around the circumference of the patient's head.

13. The apparatus recited in claim 1, wherein the medical device comprises a rigid body for supporting one or more target fiducials for registering the patient's head in an image guided surgical procedure.

14. The apparatus recited in claim 1, wherein the medical device comprises a surgical tool for performing a surgical procedure on the patient.

15. The apparatus recited in claim 1, further comprising a bracket connected to the support structure, the bracket being configured to cooperate with a patient support structure to help stabilize the position of the patient.

16. The apparatus recited in claim 1, wherein the support structure comprises C-shaped frame for extending laterally around the patient's head across the forehead and wrapping at least partially around the back of the head.

17. The apparatus recited in claim 16, wherein the support device further comprises a top member connected to the middle of the C-shaped frame, the top member extending arcuately up and over the top of the patients head, ends of the C-shaped frame and a free end of the top member being interconnected by a flexible elongated member.

18. The apparatus recited in claim 1, further comprising at least one device mount connected to the support structure, the at least one device mount being adapted to provide a releasable connection with the medical device.

19. The apparatus recited in claim 1, wherein the jamming structure comprises a substrate and a plurality of sheets supported by the substrate and arranged in an adjacent and overlying manner.

20. The apparatus recited in claim 19, wherein the sheets extend generally vertically from the substrate.

21. The apparatus recited in claim 19, wherein the sheets extend generally horizontally from one or more posts mounted to the substrate.

22. The apparatus recited in claim 19, wherein the sheets comprise sheets of paper or plastic.
